(19) United States
(12) Patent Application Publication
(10) Pub. No.: US 2014/0031642 A1
(43) Pub. Date: Jan. 30, 2014

(54) INTRA BODY CAPSULE MOTION SENSING AND POSITION DETERMINATION SYSTEMS AND METHODS

(71) Applicants: Yoav Kimchy, Haifa (IL); Anton Plotkin, Tel-Aviv (IL); Ronen Lifshitz, Misgav (IL); Oleg Obodovsky, Haifa (IL)

(72) Inventors: Yoav Kimchy, Haifa (IL); Anton Plotkin, Tel-Aviv (IL); Ronen Lifshitz, Misgav (IL); Oleg Obodovsky, Haifa (IL)

(73) Assignee: CHECK-CAP LTD., Isfiya, Mt. Carmel (IL)

(21) Appl. No.: 13/882,503

(22) PCT Filed: Oct. 31, 2011
(86) PCT No.: PCT/IB2011/002926

§ 371 Date: Oct. 16, 2013

Related U.S. Application Data
(60) Provisional application No. 61/344,867, filed on Oct. 29, 2010.

(51) Int. Cl. A61B 6/12

(52) U.S. Cl. CPC A61B 6/12
USPC 600/302; 600/409; 600/424

(57) ABSTRACT

A system for detecting clinically-relevant features of a gastrointestinal (GI) tract of a subject. The system comprises an external magnetic field generator adapted to be affixed to a patient for generating at predetermined time intervals a magnetic field with respect to the subject. The magnetic field has a predetermined polarity and flux density, thereby establishing a patient coordinate system based upon the magnetic field. The system includes a capsule adapted to be swallowed by a subject. The system further includes at least one radiation source emitting X-ray or gamma radiation and at least one radiation detector. The radiation detector is configured to detect in a first energy window collimated X-ray fluorescence radiation from the X-ray contrast agent composition excited by the emitted radiation, and to detect a second energy window Compton-backscattered radiation from the X-ray contrast agent and the wall of the GI tract produced in response to the emitted radiation. The system includes a magnetic field detector configured to generate a first output responsive to a detected polarity and detected levels of flux density during exposure to the externally generated magnetic field. A processor generates a second output based upon relative changes in the first output, the first output varying as a function of relative changes in physical location or angular orientation of the capsule associated with movement of the capsule within the GI tract. A control unit configured to selectively enable or disable X-ray or gamma ray emissions from the capsule produced by the at least one radiation source based upon the second output, such that the GI tract is selectively irradiated upon detected movement of the capsule.

### CROSS-REFERENCE TO RELATED APPLICATIONS

This application claims the benefit of priority of U.S. provisional application No. 61/344,867, entitled “Intra Body Capsule Motion Sensing and Position Determination,” filed on Oct. 29, 2010, the contents of which are incorporated herein by reference in their entirety.

### TECHNICAL FIELD

The present disclosure is directed generally to detecting motion of a capsule within the gastrointestinal tract of a patient and activating the capsule only when certain movements occur. More particularly, the present invention is directed to utilizing the correlation of capsule movement in the colon and a change in angular orientation as an indication for capsule movement in the colon. In addition, systems and methods for tracking changes in the position of the capsule are described as well as systems and methods for locating the position of the capsule in the patient frame of reference.

### BACKGROUND

Colorectal cancer is one of the leading causes of death in the Western world. Clinical evidence suggests that early detection of primary colorectal cancer leads to a 90% or better 5-year survival rate, while detection of the disease when it has already metastasized leads to poor prognosis with a 50% or less 5-year survival rate and a 30% recurrence rate. Colorectal cancer screening and early detection have a substantial positive impact on the prognosis of this malignancy.

Some systems and methods directed at the detection of polyps and other clinically-relevant features that may harbor the potential for cancer of the gastrointestinal (GI) tract, particularly colorectal cancer, are discussed in U.S. Pat. No. 7,787,926, the contents of which are incorporated herein by reference. These systems and methods include a capsule designed to be swallowed by a patient and to travel through the GI tract. In a typical case, the trip can take between 24-48 hours after which the imaging capsule exits in the patient's feces. For example, a subject swallows a contrast agent, and, typically after a waiting period, a capsule comprising one or more gamma and/or X-ray radiation sources and radiation detectors. As the capsule travels through the GI tract, the radiation sources “illuminate” the vicinity of the capsule. The GI contents (including the contrast agent), GI wall, and tissue outside of the GI tract act as a scattering media for the emitted radiation, typically primarily through the process of Compton scattering. The scattered photons then travel back through the GI contents, which include the contrast agent. The radiation detectors count appropriately Compton-backscattered photons and transmit the count rate information to an external recording unit worn by the subject.

The count rates collected by each detector per unit time interval are analyzed, typically only for predetermined photon energy windows. These data are presented to a physician in a manner that enables him to assess the likelihood that there is a polyp or some other anatomical deformation in the GI tract. In some embodiments, the data are also analyzed to indicate a general area of the colon where such a deformation may exist. These polyps or anatomical anomalies may be the result of a tumor beginning to grow within the GI tract. If the physician suspects the presence of a polyp or some other anatomical anomaly that may be cancerous or pre-cancerous, the subject is typically referred for further diagnostic testing, such as colonoscopic examination.

Because the aforementioned process involves radiation of the human body, and because excessive radiation can have potentially adverse consequences on the patient, it is desirable to minimize the amount of time that the capsule is activated, that is, illuminating with radiation. Therefore, in such systems it is preferable to activate the capsule for scanning only when the capsule is moving through the GI tract. That is, it is not preferable to keep the capsule activated for scanning while it is stationary in the GI tract.

FIG. 6 is a graph illustrating an example of a tracing of the position coordinates and the angular coordinates of a capsule in a patient. As detected by the magnetic tracking system and the accelerometer on board the capsule, movement in the XYZ trace can be seen to be correlated with a change in the angular orientation of the capsule as well as its relative position in the patient over time. Pressure changes are also measured in the capsule.

In a clinical trial conducted by Check-Cap Ltd in January 2010, it was shown that there is very high correlation between capsule movements in the colon and change in angular orientation of the capsule as it was moving in the colon. This change in angular orientation was monitored by a magnetic tracking system (Motilis, Switzerland) that monitored a magnet that was embedded in the capsule. In addition, a 3D accelerometer (ST electronics) monitored the direction of the gravitational force vector. Both systems showed clear angular change of direction during movements of the capsule in the colon.

It may therefore be desirable to provide systems and methods for sensing capsule movement in the GI tract and to activate the capsule, which in turn starts the capsule scanning, only upon sensing such movement. Capsule movement in the GI tract may be sensed by monitoring capsule movements in the colon and change in angular orientation of the capsule. Systems and methods of the disclosure utilize the correlation of capsule movement, which may occur due to changes in the relative location of the capsule as it transits the GI tract, as well as changes in the relative angular orientation of the capsule as it undergoes rotation within the GI tract. Consequently, detecting either event serves as a more reliable indication of actual capsule movement within the colon. In addition, systems and methods for tracking changes in the position of the capsule are described as well as systems and methods for locating the position of the capsule in the patient frame of reference.

### SUMMARY OF THE INVENTION

In various aspects, the present disclosure is directed to systems for detecting clinically-relevant features of a gastrointestinal (GI) tract of a subject. According to some aspects, the system may comprise an external magnetic field generator adapted to be affixed to a patient for generating at predetermined time intervals a magnetic field with respect to the subject. The magnetic field may have a predetermined polarity and flux density, thereby establishing a patient coordinate system based upon the magnetic field. A capsule adapted to be swallowed by a subject may include a radiation source emitting X-ray or gamma radiation and a radiation detector. In some aspects, the radiation detector may detect in a first energy window collimated X-ray fluorescence radiation from the X-ray contrast agent composition excited by the emitted radiation, and in a second energy window Compton-backscattered radiation from the X-ray contrast agent and the wall of the GI tract produced in response to the emitted radiation. In accordance with various aspects, a magnetic field detector may generate a first output responsive to a detected polarity and detected levels of flux density during exposure to the externally generated magnetic field. A processor may generate a second output based upon relative changes in the first output. The first output may vary as a function of relative changes in physical location or angular orientation of the capsule associated with movement of the capsule within the GI tract. The system may include a control unit configured to selectively enable or disable X-ray or gamma ray emissions from the capsule produced by the a radiation source based upon the second output, such that the GI tract is selectively irradiated upon detected movement of the capsule.

In accordance with some aspects, the radiation source may include an X-ray generator and a power source, and the control unit selectively enables or disables radiation emissions by regulating power supplied to the X-ray generator based upon the second output.

According to some aspects, the radiation source may comprise a radioisotope selected from the group comprising T1201, Xe133, Hg197, Yb169, Ga67, Tc99, Tc99m, In111, I131 and Pd100.

In accordance with some aspects, the control unit may include a radiation shield movably configured with respect to the radiation source, and a motor configured to controllably reposition the radiation shield relative to the radiation source, such that the GI tract is selectively irradiated based upon the second output.

According to various aspects, the magnetic field detector may comprise a magnetic compass.

In accordance with various aspects of the disclosure, the external magnetic field generator may comprise magnets affixed at spaced apart locations on a belt to be worn by the subject.

In some aspects, the magnets may comprise electromagnets and a controlled current generator.

In accordance with some aspects, the controlled current generator varies the current supplied to each of the electromagnets.

In various aspects, the present disclosure is directed to a system for detecting clinically-relevant features of a gastrointestinal (GI) tract of a subject. The system may comprise an external signal transmitter adapted to be affixed to a patient for generating at predetermined time intervals a transmission field with respect to the subject. The transmission field may have a predetermined amplitude and frequency, thereby establishing a patient coordinate system based upon the transmission field. A capsule adapted to be swallowed by a subject may include a radiation source emitting X-ray or gamma radiation and a radiation detector. In some aspects, the radiation detector may be configured to detect in a first energy window collimated X-ray fluorescence radiation from the X-ray contrast agent composition excited by the emitted radiation, and to detect in a second energy window Compton-backscattered radiation from the X-ray contrast agent and the wall of the GI tract produced in response to the emitted radiation. In accordance with some aspects, a transmission field detector may generate a first output responsive to a detected amplitude and frequency during exposure to the externally generated transmission field. The system may further comprise a processor configured to generate a second output based upon relative changes in the first output, the first output varying as a function of relative changes in physical location or angular orientation of the capsule associated with movement of the capsule within the GI tract. The system may include a control unit configured to selectively enable or disable X-ray or gamma ray emissions from the capsule produced by the radiation source based upon the second output, such that the GI tract is selectively irradiated upon detected movement of the capsule.

In accordance with some aspects, the external signal transmitter comprises a transducer configured to generate radio frequency emissions.

According to various aspects, the transmission field detector further comprises an antenna configured to receive said radio frequency emissions.

In accordance with various aspects of the disclosure, the external signal transmitter comprises a transducer configured to generate ultrasound emissions.

In some aspects, the transmission field detector further comprises a transducer configured to receive said ultrasound emissions.

The present invention will be more fully understood from the following detailed description of embodiments thereof, taken together with the drawings.

### BRIEF DESCRIPTION OF THE DRAWINGS

In the drawings,

FIG. 1A is a schematic illustration of an exemplary screening system in accordance with various aspects of the disclosure;

FIG. 1B is a schematic illustration of an exemplary capsule of the system of FIG. 1A in accordance with various aspects of the disclosure;

FIG. 1C is a schematic illustration of an exemplary external data-recording unit of the system of FIG. 1A in accordance with various aspects of the disclosure;

FIG. 2A is a schematic illustration of an exemplary screening system in accordance with various aspects of the disclosure;

FIG. 2B is a schematic illustration of an exemplary capsule of the system of FIG. 2A in accordance with various aspects of the disclosure;

FIG. 2C is a schematic illustration of an exemplary belt of the system of FIG. 2A in accordance with various aspects of the disclosure;

FIG. 2D is a schematic illustration of an exemplary screening system in accordance with various aspects of the disclosure;

FIG. 2E is a schematic illustration of an exemplary magnet of the system of FIG. 2D in accordance with various aspects of the disclosure;

FIG. 2F is a schematic illustration of an exemplary screening system in accordance with various aspects of the disclosure;

FIG. 2G is a schematic illustration of an exemplary electromagnet of the system of FIG. 2D in accordance with various aspects of the disclosure;

FIG. 3A is a schematic illustration of an exemplary screening system in accordance with various aspects of the disclosure;

FIG. 3B is a schematic illustration of an exemplary capsule of the system of FIG. 3A in accordance with various aspects of the disclosure;

FIG. 3C is a schematic illustration of an exemplary belt of the system of FIG. 3A in accordance with various aspects of the disclosure;

FIG. 3D is a schematic illustration of an exemplary screening system in accordance with various aspects of the disclosure;

FIG. 3E is a schematic illustration of an exemplary adhesive member of the system of FIG. 3D in accordance with various aspects of the disclosure;

FIG. 4A is a schematic illustration of an exemplary screening system in accordance with various aspects of the disclosure;

FIG. 4B is a schematic illustration of an exemplary capsule of the system of FIG. 4A in accordance with various aspects of the disclosure;

FIG. 5A is a schematic illustration of an exemplary screening system in accordance with various aspects of the disclosure;

FIG. 5B is a schematic illustration of an exemplary capsule of the system of FIG. 5A in accordance with various aspects of the disclosure;

FIG. 5C is a schematic illustration of an exemplary adhesive member of the system of FIG. 5A in accordance with various aspects of the disclosure; and

FIG. 6 is a graph illustrating the tracing of the position coordinates and the angular coordinates of a capsule in the GI tract of a patient.

### DETAILED DESCRIPTION

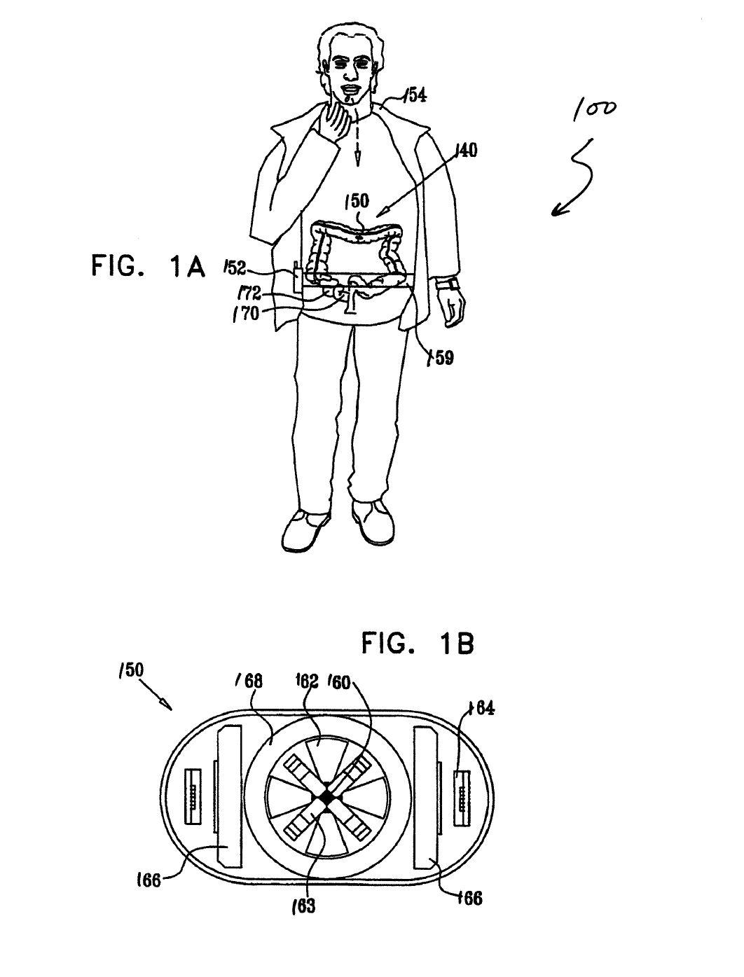

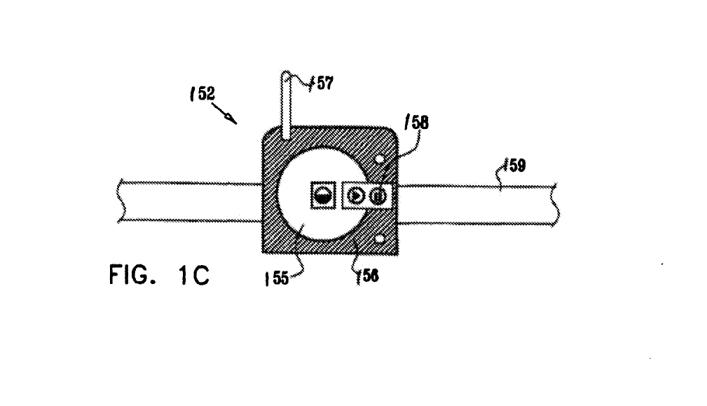

Reference is made to FIG. 1A, which is a schematic illustration of a screening system 140, in accordance with various aspects of the disclosure. The system 140 typically comprises an ingestible capsule 150 and an external data-recording unit 152. For some applications, the data-recording unit 152 may be worn on the waist of a subject 154 (as shown in FIG. 1A) or elsewhere on the subject's body, such as the wrist (configuration not shown), etc. Alternatively, for some applications, the capsule 150 may comprise an internal data-recording unit, and the external data-recording unit 152 may not be provided. In these applications, the data recorded by the capsule 150 is retrieved after the capsule has been expelled from the body.

Reference is now made to FIG. 1B, which is a schematic illustration of an exemplary capsule 150, in accordance with various aspects of the present disclosure. The capsule 150 may comprise at least one radiation source 160 adapted to emit gamma rays, X-rays, and/or beta electrons (i.e., radiation having an energy of at least 10 keV), at least one gamma and/or or X-ray radiation detector 162, and, typically, at least one collimator 163 adapted to collimate the radiation produced by the radiation source 160. For some applications, the radiation source 160 may comprise a radioisotope or a miniature radiation generator.

In some aspects of the disclosure, radiation source 160 may comprise a miniature X-ray generator, such as those described in one or more of the following references: U.S. Pat. Nos. 6,134,300 and 6,353,658 to Trebes et al.; Haga, A. et al., “A miniature x-ray tube,” Applied Physics Letters 84(12):2208-2210 (2004); and Gutman, G. et al., “A novel needle-based miniature x-ray generating system,” Phys Med Biol 49:4677-4688 (2004). Such a miniature X-ray generator or X-ray tube may be used for radiation source 160 instead of a radioisotope to illuminate the colon contents with X-ray photons. Turning such a generator on and off as needed typically reduces exposure of the subject to radiation. In addition, the energy range can be better controlled and the flux may be higher for the on periods without increasing subject total exposure.

According to various aspects, the capsule 150 may comprise one or more gamma and/or X-ray radiation sources and/or sources of beta electrons, such as T1201, Xe133, Hg197, Yb169, Ga67, Tc99, Tc99m, In111, I131 or Pd100. With the use of such radioisotopes as a radiation source, however, it is desirable to incorporate a radiation shield which can be selectively moved relative to the radiation source, thereby limiting irradiation of the GI tract only when needed, such as during periods when movement of the capsule within the GI tract has been affirmatively detected.

The capsule 150 also typically comprises circuitry 164 (which, for some applications, includes a pressure sensor), a power supply 166, such as a battery, a wireless communication device 167 for communicating with external data-recording unit 152 (communication device not shown), and a radiation shield 168. The shield 168 may comprise a material having a high atomic weight and a high specific density, such as lead, tungsten, tantalum, or gold. In an embodiment of the present invention, the radiation source 160 and detector 162 are arranged to “observe” the entire 4 pi squared sphere (or a portion of it) surrounding the capsule. According to some aspects, the capsule incorporates an electromechanical control unit, comprising the radiation shield 168 and the radiation source 160 which are movable relative to one another via a low power actuator, such as a motor and power source (not shown), thereby selectively permitting and preventing exposure of GI lumen tissue by emissions from the source 150 based on their relative positions. For example, in some aspects, the source 160 may be stationary and the shield 168 movable. In some aspects, the shield 168 may be stationary and the source 160 movable. In some aspects, the shield 168 and the source 160 may both be movable. In the case where the radiation source comprises an electrically-powered X-ray generator, the control unit comprises a regulated power source which is activated when emissions are desired and deactivated when emissions are not desired, such as when the capsule is stationary.

Reference is now made to FIG. 1A. During a typical screening procedure using system 140, an oral contrast agent 170 is administered to subject 154. Contrast agent 170 is typically adapted to pass through a gastrointestinal (GI) tract 172 and be expelled with the feces, substantially without being absorbed into the blood stream. The contrast agent material may be similar to compounds used routinely for the study of the GI with X-rays, such as Barium sulfate liquid concentrate, iodine-based compounds, or other such materials. For some applications, additional appropriate contrast agents include Tantalum, Gadolinium, Thorium, Bismuth, and compounds of these materials. After the contrast agent is administered (e.g., several hours after the contrast agent is administered), subject 154 swallows capsule 150.

Capsule 150 travels through GI tract 172, emitting gamma and/or X-ray radiation. Beginning at a certain point in time, capsule 150 records the Compton scattered gamma and/or X-ray photons that strike radiation detectors 162. The count rate information received from each of the radiation detectors is typically stored together with a time stamp for that measurement. Within a time period typically of less than one second (e.g., several tens to several hundred milliseconds), it is assumed that the capsule and the surrounding colon wall and the contrast agent are in quasi-steady state. Taking small enough time intervals and integrating the counts over the small intervals allows for this quasi-steady-state assumption. The data may be stored in the capsule and sent by the capsule to the external recording unit from time to time, or after data-gathering has been completed.

Reference is now made to FIG. 1C, which is a schematic illustration of the external data-recording unit 152, in accordance with an exemplary embodiment of the present disclosure. The data-recording unit 152 may comprise a receiver/memory unit 155, a support electronics/battery unit 156, an antenna 157, and/or user controls 158. In some aspects, the unit 152 may also include a strap 159, such as a belt or wrist/arm strap, for coupling the unit to the subject 154.

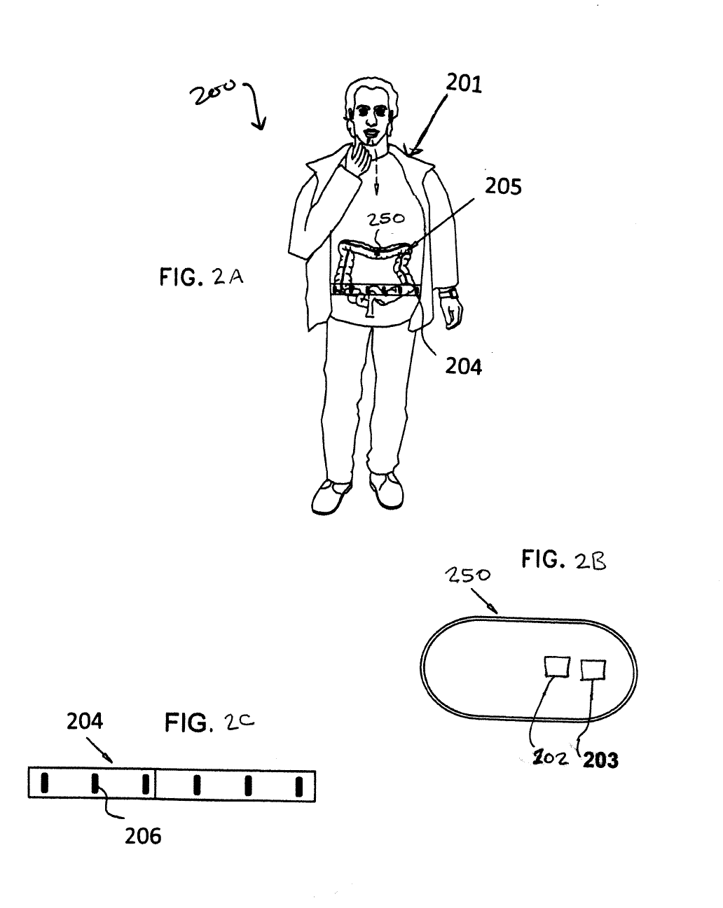

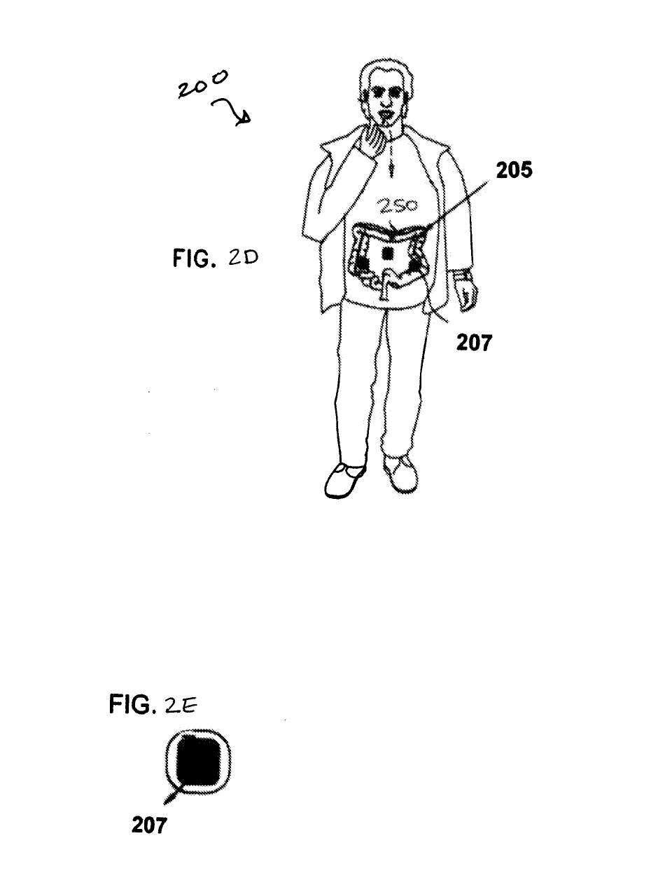

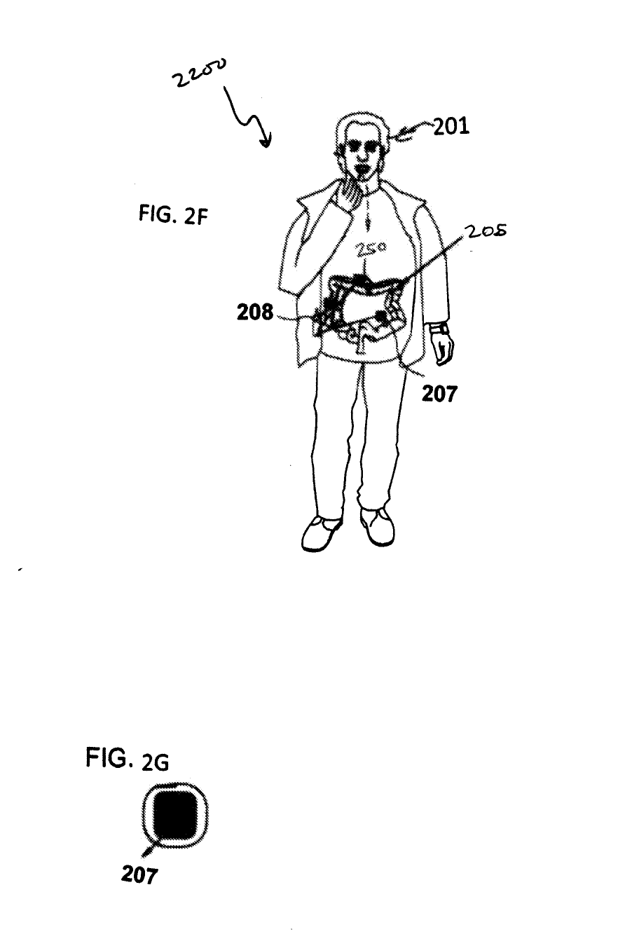

Referring now to FIG. 2A, an exemplary screening system 200 includes an exemplary capsule 250, which is some aspects may be similar to the capsule 150 described above. The capsule 250 is sized and adapted to be swallowed by a patient 201 for diagnostic or treatment purposes. As shown in FIG. 2B, the capsule 250 incorporates a magnetic field detector comprising a miniature magnetic compass 202 (such as, for example, YAS529 from Yamaha) and a processor 203 that is communicable with the magnetic compass 202. For clarity of illustration, the capsule 250 is shown without the components described above with reference to capsule 150. Referring now to FIG. 2C, an exemplary belt 204 according various aspects of the disclosure is shown. The belt 204 may include one or more magnets 206, such as, for example, permanent magnets or electromagnets, placed thereon. Additionally or alternatively, in some aspects, the magnet(s) 206 can be attached to an article of clothing, such as a vest, or can be attached to the patient 201 by an adhesive sticker, for example, as discussed below in connection with FIG. 2D. It should be appreciated that there are numerous conventional ways the magnets 206 can be coupled with a patient 201. In each case, the magnets 206 are positioned so that they are securely attached to the patient 201 and move with the patient 201. This fixed positioning defines the magnets 206 in a patient coordinate system/frame of reference.

FIG. 2D illustrates an exemplary screening system 1200 including a patient 201 wearing one or more exemplary sticker magnets 207 and having swallowed the exemplary capsule 250. An exemplary sticker magnet 207 is shown in FIG. 2E. FIG. 2F illustrates an exemplary screening system 2200 including a patient 201 wearing an exemplary sticker electromagnet 207 and having swallowed the exemplary capsule 250. An exemplary sticker electromagnet 207 is shown in FIG. 2G.

In use, referring to FIGS. 2A-2E, a subject swallows a contrast agent, and, typically after a waiting period, swallows the capsule 250. Eventually, after some time, the capsule reaches the colon of the GI tract. As the capsule 250 travels through the GI tract, the radiation sources of the capsule 250 “illuminate” the vicinity of the capsule 250 to enable imaging of the tract. When the capsule 250 stops moving and comes to rest, the capsule 250 may be deactivated to cease irradiating the GI tract, thereby avoiding unnecessary radiation of the patient and limiting potential side effects of the screening process.

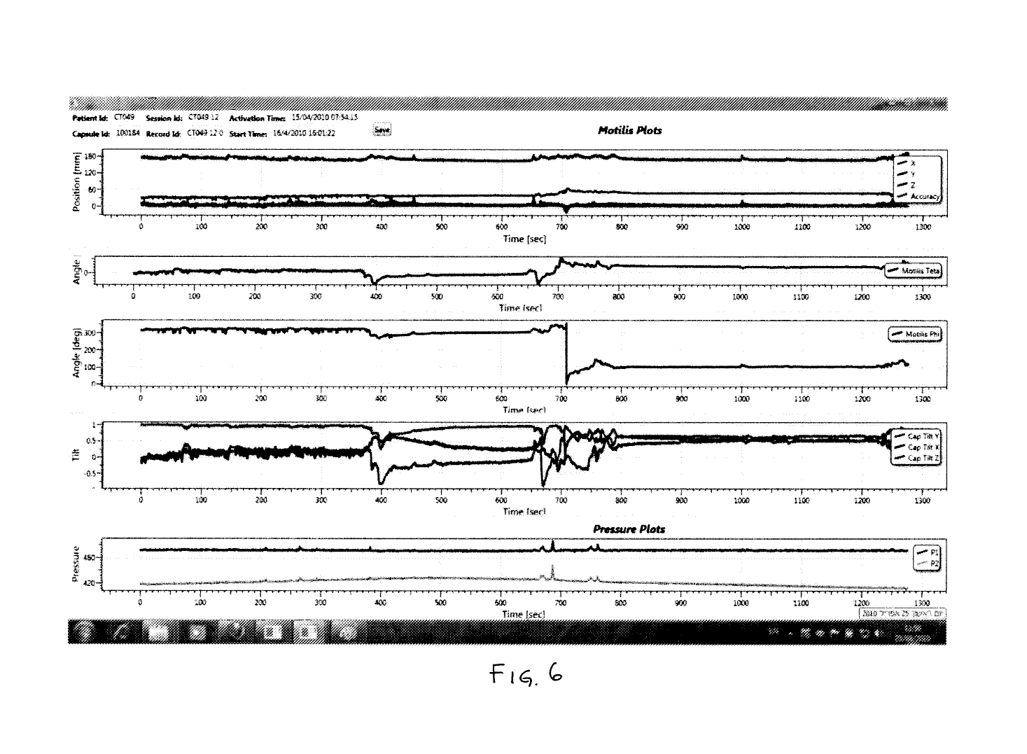

When the exemplary capsule 250 in the colon 205 starts to move, it performs a change in orientation as described above with reference to FIG. 6. The change in orientation of the capsule changes the orientation of the magnetic field (or fields, if a few magnets 207 are detected simultaneously by the magnetic compass 202) that the magnetic compass 202 in the capsule 250 detects. An algorithm of instructions programmed into the capsule processor 203 calculates the difference between the previous orientation of the capsule 250 and the current orientation of the capsule 250, sampling in short time intervals, for example, typically from a few milliseconds to a few seconds. If a predefined threshold of capsule orientation change is reached, the processor 203 determines that the capsule 250 has changed orientation considerably from the last orientation and movement of the capsule is thus about to occur.

According to some aspects, an adaptive algorithm is employed in the capsule 250 to adapt to changes and the level of changes in orientation of the capsule 250. According to some aspects, the algorithm can be implemented in a unit external to the capsule 250, which is configured to communicate with the capsule 250.

Referring now to FIGS. 2F and 2G, according to some aspects, the system 2200 includes electromagnets 207 and the capsule 25 0 is communicable with a controlled current generator 208 for controlling the current of the one or more electromagnets 207 to vary the field of the electromagnet(s) 207. Controlling the current of the electromagnet(s) 207 allows the algorithm to modify the magnetic flux of the electromagnet(s) 207 such that when the capsule 250 is far inside the body not close to the electromagnet(s) 207 on the patient 201, the magnetic field can be intensified by applying more current to the electromagnet(s) 207, thereby improving the sensitivity of the compass 202 within the capsule 250. When the capsule 250 comes near the electromagnet 207, it may be advantageous to reduce the electric current in the electromagnet 207 to reduce the magnetic flux, in order to prevent saturation of the magnetic compass 202 in the capsule 200. In addition, the electromagnet(s) 207 can be switched on and off depending on the position of the capsule 250, thus optimizing in real time the magnetic field that the capsule compass 202 senses. This further improves the sensitivity of the capsule 25 0 for motion detection.

It should be appreciated that, according to some aspects, the decision as to whether the capsule 250 has moved is based on a correlation of at least one parameter coming from at least one sensor such as a pressure sensor, a strain gauge on the capsule 250, a capacitive sensing on the capsule 250, an accelerometer, and other parameters coming from the imaging data that the capsule 250 generates.

According to some aspects, the electromagnets 207 are switched on one at a time. In response, the capsule 250 transmits the detected direction of the magnetic compass 202 relative to the magnetic field. Applying such a sequence allows the receiver (or the capsule 250) to gather information about the position of the capsule 250 by calculating the point of intersection of the directions to the magnetic field that were sensed by the capsule 250 and combines this information with the position of the external electromagnets 207 on the patient 201. This enables the position of the capsule 250 in the patient frame of reference to be calculated.

In another embodiment of this invention, the capsule 250 senses the switching of the external magnet and acts as in a UART (Universal Asynchronous Receive Transmit) port protocol to lock on the switching of the magnets and transmit relative orientation of the magnetic compass 202 to the magnet 207 that is on after the transit of the switching to the next magnet 207 has occurred. In this way, there is no need for RF protocol to be involved in the synchronization of the transmission of direction after magnet 207 switching.

This exemplary procedure of switching each magnet 207 in its turn or switching a subset of the magnets 207 or modulating their current in a known way to manipulate the direction of the magnetic force vector detected by the capsule magnetic sensor can be repeated from time to time and allows tracking the capsule 250 in its movement in the GI tract.

According to some aspects, a single, external fixed-field magnet (not shown) can be used to approximately locate the position of the capsule 200. The fixed-field magnet can be brought near the body of the patient 201 and the capsule 250 can continuously send the direction of the magnetic “North” it is detecting via the magnetic compass 202 and the amplitude of the magnetic moment, so that the closest point to the capsule 250 from the outside can be determined.

According to some aspects, magnets 207 on the patient 201 are placed with opposite pole direction. As the capsule 250 moves along the gastrointestinal tract, the magnetic force can be registered and the approximate position of the capsule 250 can later be deduced from the change of direction of the magnetic field as the capsule 250 moves to “see” one magnetic pole and then sensing the change to the opposite pole near a different magnet along the route of the capsule 250.

The influence of Earth's magnetic field may affect the aforementioned procedures. Two mechanisms can be used to compensate for this influence. First, with no permanent magnets on the body of the patient 201 (or with all the electro magnets switched off) the direction of the earth magnetic field can be determined and later compensated for when magnets on the patient 201 body are applied (or switched on). Second, the use of relatively strong magnets (typically a few hundred to a few thousand Gauss in relation to 0.5 Gauss of Earth's magnetic field) means that the Earth's magnetic influence on the direction of the capsule's internal magnetic compass 202 will be minimal when magnets are placed (or switched on) the patient 201.

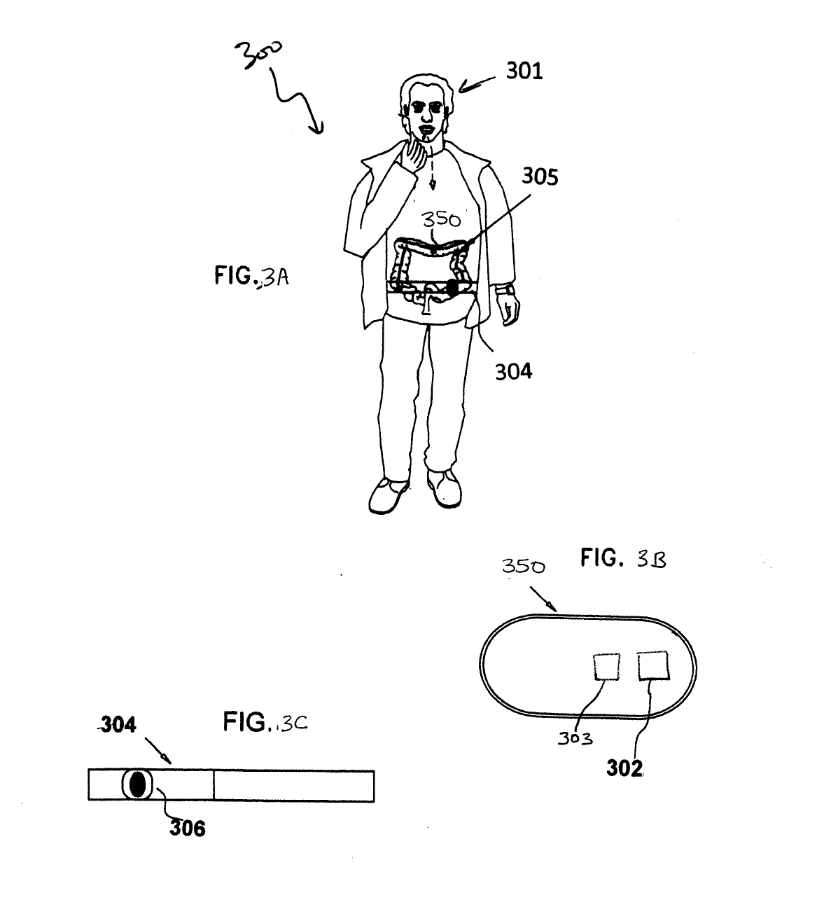

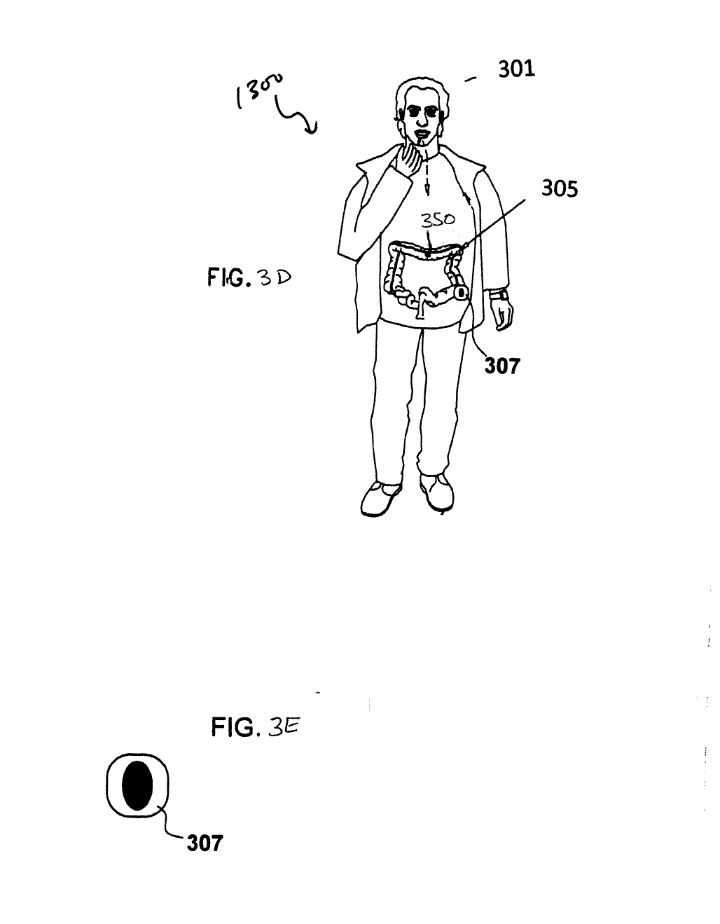

Referring now to FIGS. 3A-3C, an exemplary embodiment of another method of detecting motion of a capsule within the Gastro Intestinal tract of a patient and activating the capsule only when such movements occur includes use of an alternating magnetic system to sense a change in orientation and/or position of the capsule in the colon and hence capsule movement. In some aspects, such an alternating magnetic system may be similar to a magnetic position sensing such as those manufactured by Ascension http://www.ascension-tech.com/ and Biosense http://www.biosensewebster.com/products/navigation/.

For example, as illustrated in FIG. 3A, an exemplary screening system 300 includes an exemplary capsule 350, which is some aspects may be similar to the capsule 150 described above. The capsule 350 is sized and adapted to be swallowed by a patient 301 for diagnostic or treatment purposes. As shown in FIG. 3B, the capsule 350 includes a coil 302, such as, for example, a miniature 3D coil, and a processor 303. The 3D coil may comprise three coils that are each wound at 90 degrees relative to the other two coils, respectively. The processor 303 is communicable with the coil to pick up signals from the three channels of the coil. Referring now to FIG. 3C, an exemplary belt 304 according various aspects of the disclosure is shown. The belt 304 may include one or more transmitters 306 such as, for example, a 3D coil with each coil at 90 degrees relative to the other two coils). In some aspects, the belt may include electromagnets. It should be appreciated that the transmitter 306 may attached to a vest to be worn by a patient, or may be attached to a patient by an adhesive sticker 307 (FIG. 3E). In any event, the one or more transmitter(s) are positioned so that they are securely attached to the patient and move with the patient, thus defining the transmitters in the patient system of coordinates.

In use, when the exemplary capsule 350 in the colon starts to move, a change in orientation occurs, as described above relative to FIG. 6. The change in orientation of the capsule 350 in turn changes the orientation of the 3D coil 302 in the capsule 350 relative to the transmitter 306, for example, an external 3D coil. The change in orientation of the capsule 3D coil 302 relative to the transmitter 306 changes the magnetic flux detected in the coils, and the processor 303 in the capsule 350 can compute and detect the change in orientation of the capsule. Change in orientation is usually associated with capsule movement, so that an algorithm in the capsule can use this information to trigger capsule scanning due to movement.

According to some aspects, the processor 303 on the exemplary capsule 350 calculates the change in position due to change in magnetic flux detected by the 3D coil 302 on board the capsule 350. The changes related to position movement are used by the processor 303 to determine when to start and stop scanning by the capsule 350. Additionally, relative position information which is correlated to magnetic flux amplitude in the 3D coil 302 on board the capsule 350 can be transmitted from the capsule 350 to an external receiver (not shown) or stored in the capsule 350 for later analysis of the position of the capsule 350 in time relative to the external 3D coil transmitter 306 on the patient 301. The calculation as to whether the capsule 350 has moved is based on a correlation of at least one parameter coming from at least one sensor such as a pressure sensor, an accelerometer, an alternating magnetic detector, and other parameters coming from imaging data the capsule generates.

In another exemplary embodiment of a screening system including an alternating magnetic system, referring again to FIG. 3A, the capsule 350 may include at least one coil and an electronic circuit that is in communication with the coil to pick up signals from the coil. On the patient, a transmitter coil or a number of transmitter coils or electromagnets are placed on a belt or vest or are attached to the patient by an adhesive sticker. The transmitter(s) is positioned so that it is well attached to the patient and moves with the patient, thus defining the transmitter(s) in the patient system of coordinates.

When the capsule 350 in the colon 305 starts to move, it may change orientation or position. This in turn changes the magnetic flux picked up by the coil, and the controller in the capsule can compute and detect change in position or orientation of the capsule. In some aspects, the decision on whether the capsule has moved is based on a correlation of a few parameters coming from a few sensors such as a pressure sensor, an accelerometer, the alternating magnetic detector, and other parameters coming from the imaging data that the capsule generates.

In another exemplary embodiment of a screening system including an alternating magnetic system, referring again to FIG. A, the capsule 350 incorporates at least one coil and an electronic circuit that is in communication with the coil to transmit signals from the coil. On the patient a receiver coil or a number of receiver coils are placed on a belt, vest or attached to the patient by an adhesive sticker. The receiver(s) is positioned so that it is well attached to the patient and moves with him, thus defining the receiver(s) in the patient system of coordinates.

When the capsule 350 in the colon 305 starts to move, it may change orientation or position. This in turn changes the magnetic flux picked up by the coils on the patient body. This information is processed by electronic circuits and a controller on the patient body and is used to compute if the capsule has moved. This information in turn is transmitted to the capsule, and the controller in the capsule can compute and detect change in position or orientation of the capsule.

In another exemplary embodiment of a screening system including an alternating magnetic system, referring again to FIG. 3A, the capsule 350 incorporates at least one magnetic sensor such as a fluxgate magnetometer (e.g., FLC3-70 from Stefan Mayer Instruments) or MI magnetometer (e.g., MI-CB-1DK from Aichi Steel Ltd. Japan) and an electronic circuit that is in communication with the magnetometer to pick up signals. On the patient, a transmitter coil or a number of transmitter coils or electromagnets are placed on a belt or vest or attached to the patient by an adhesive sticker. The transmitter(s) is positioned so that it is well attached to the patient and moves with him, thus defining the transmitter(s) in the patient system of coordinates.

When a capsule in the colon starts to move, the capsule may change orientation or position, which in turn changes the magnetic flux picked up by the magnetometer, and the controller in the capsule can compute and detect change in position or orientation of the capsule. The decision on whether the capsule has moved is based on a correlation of a few parameters coming from a few sensors such as a pressure sensor, an accelerometer, the alternating magnetic detector, and other parameters coming from the imaging data that the capsule generates.

In another exemplary embodiment of a screening system including an alternating magnetic system, referring again to FIG. 3A, the capsule 350 incorporates at least one coil and an electronic circuit that is in communication with the coil to transmit signals from the coil. On the patient, a magnetometer or a number of magnetometers (such as FLC3-70 from Stefan Mayer Instruments) magnetometer or MI magnetometer (e.g., MI-CB-1DK from Aichi Steel Ltd. Japan) are placed on a belt or vest or attached to the patient by an adhesive sticker. The magnetometer(s) is positioned so that it is well attached to the patient and moves with him, thus defining the magnetometer(s) in the patient system of coordinates.

When a capsule in the colon starts to move, it may change orientation or position, which in turn changes the magnetic flux picked up by the magnetometer(s) on the patient's body. This information is processed by electronic circuits and a controller on the patient body and is used to compute if the capsule has moved. This information in turn is transmitted to the capsule, and the controller in the capsule can compute and detect change in position or orientation of the capsule. The decision on whether the capsule has moved is based on a correlation of a few parameters coming from a few sensors such as a pressure sensor, an accelerometer, the alternating magnetic detector, and other parameters coming from the imaging data that the capsule generates.

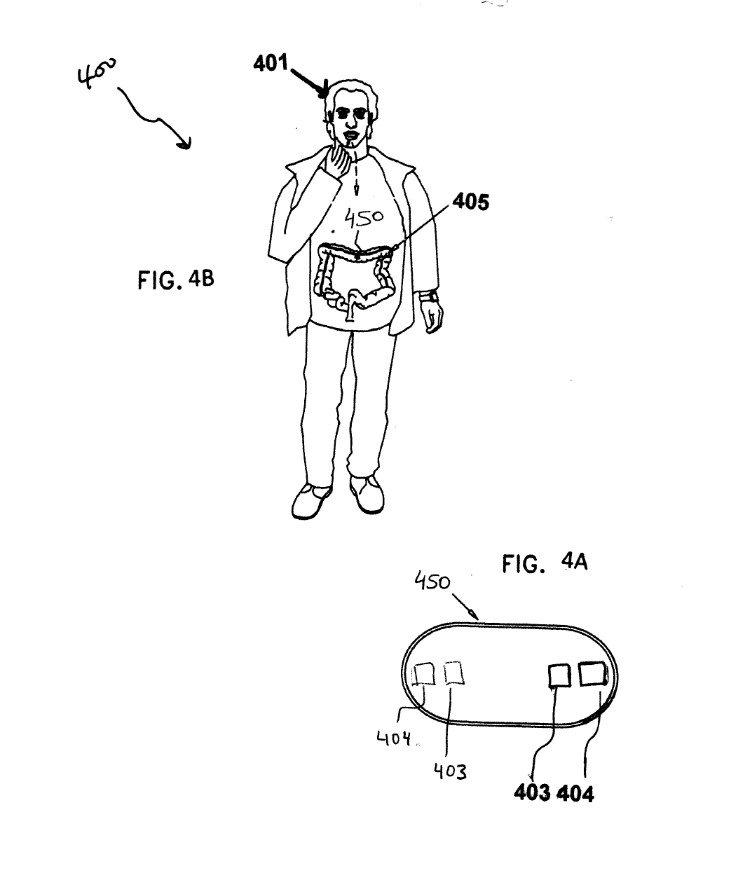

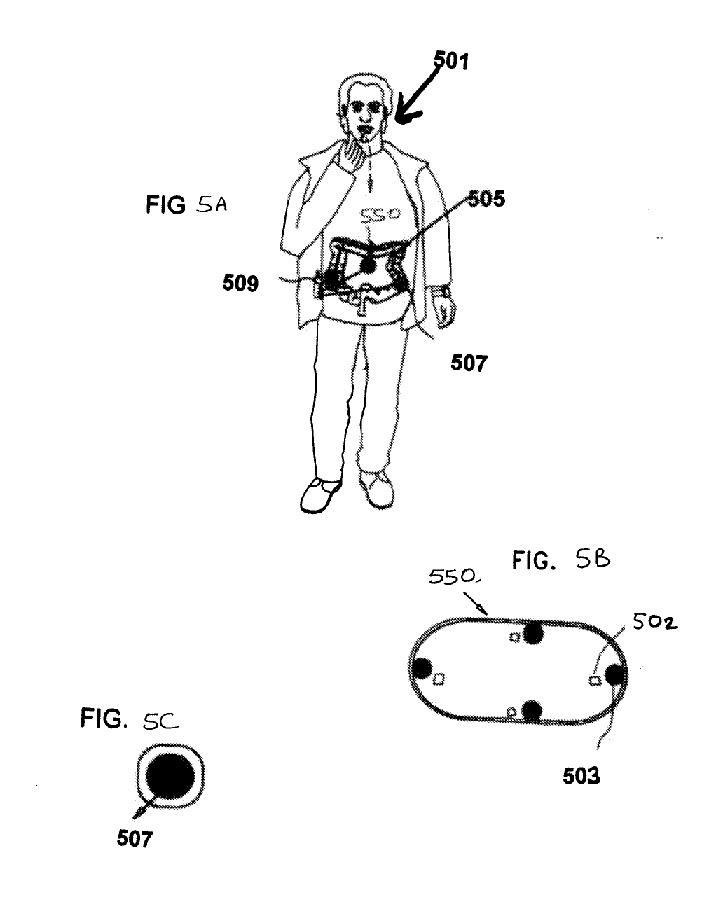

Referring now to FIG. 4A, an exemplary embodiment of another system 400 and method of detecting motion of an exemplary capsule 450 within the GI tract of a patient 401 and activating the capsule 450 only when such movements occur. The exemplary capsule 450, in some aspects, may be similar to the capsule 150 described above. The capsule 450 is sized and adapted to be swallowed by a patient 401 for diagnostic or treatment purposes. As shown in FIG. 4B, the capsule 450 includes includes at least one miniature accelerometer 404 and a processor 403 that is communicable with the accelerometer 404. The accelerometer(s) 404 is configured to sense change in orientation of the capsule 450 in the colon and hence capsule movement.

In use, when a capsule 450 in the colon 405 starts to move, the capsule 450 performs a change in orientation as described above in connection with FIG. 6. This change in orientation of the capsule 450 in turn changes the orientation of the accelerometer 404, which is directed towards the center of Earth gravity. This change in direction of the accelerometer 404 can signify change in orientation, which can be linked to capsule movement in the colon 405. The controller/processor 403 in the capsule 450 can use this information to decide when to start and stop scanning, that is, illuminating the GI tract with radiation.

It should be appreciated that by using more than one accelerometer, it may be possible to detect acceleration differences between the two or more accelerometers. This information can be also be used to sense if there is movement of the capsule 450, and hence an algorithm including instructions stored in the controller/processor 403, for example, in the capsule 450 can use this information to trigger capsule scanning due to capsule movement.

It should also be appreciated that by using one or more accelerometers 404 in the capsule 450 and one or more accelerometers (not shown) on the patient's body, it is possible to detect acceleration differences between the capsule 450 and the body-worn receiver accelerometers. This information can be used to sense if there is relative movement or relative change in orientation and hence, an algorithm in the capsule 450 or in an external receiver (not shown) can include instructions that use this information to trigger capsule scanning due to capsule movement.

In some aspects of the disclosure, the processor 403 in the capsule 450 calculates the change in position of the capsule 450. The changes related to position movement are used by the capsule processor 403 to determine when to start and stop scanning by the capsule 450. Additionally, relative position information which is correlated to acceleration differences detected on board the capsule 450 can be transmitted from the capsule 450 to the external receiver (not shown) or stored in the capsule 450 for later analysis of the position of the capsule in time relative to the external transmitter (not shown) on the patient 401. According to various aspects, the calculation as to whether the capsule 450 has moved is based on a correlation of at least one parameter coming from at least one sensor such as a pressure sensor, accelerometer, the alternating magnetic detector, and other parameters coming from the imaging data that the capsule 450 generates.

Referring now to FIG. 5A, an exemplary embodiment of another system 500 and method of detecting motion of an exemplary capsule 550 within the GI tract of a patient 501 and activating the capsule 550 only when such movements occur. The exemplary capsule 550, in some aspects, may be similar to the capsule 150 described above. The capsule 550 is sized and adapted to be swallowed by a patient 501 for diagnostic or treatment purposes. As shown in FIG. 5B, the capsule 550 includes an ultrasonic localization system to sense change in orientation and/or position of the capsule 550 in the colon 505 and hence capsule movement. For example, the capsule 550 includes one or more miniature ultrasonic transducers 503 tuned to receive externally generated ultrasound emissions and generate a first output responsive to the detected amplitude and frequency of such ultrasound transmissions, and a processor 502 that is communicable with the transducers 503. On the patient, a transmitting ultrasonic transducer or a number of transmitters 507, as shown in FIG. 5C, may be placed on a belt or vest, or attached to the patient 501 by an adhesive sticker, as shown in FIG. 5A. The transmitter(s) 507 is positioned so that it is securely attached to the patient 501 and moves with the patient 501, thus defining the transmitter(s) in the patient system of coordinates.

In use, when a capsule 550 in the colon 505 starts to move, a change in orientation occurs as described above, thereby changing the orientation of the ultrasonic transducers 503 in the capsule 550 relative to the external ultrasonic transmitter 507. This change in relative orientation changes the amplitude of the ultrasound signal received by the transducers 503 on the capsule 550. This change in relative amplitude received by the capsule transducers 503 can signify change in orientation of the capsule 550, which can be linked to capsule movement in the colon 505. In turn, the processor 502 will identify relative changes in the detected ultrasound emissions over time and generate a second output associated with detected movement of the capsule within the GI tract. The processor/controller 502 in the capsule 550 can use this information to decide when to start and stop scanning by the capsule, thereby limiting scanning radiation emissions to the adjacent body tissue only when needed, such as when the capsule is moving within the GI tract.

It should be appreciated that by using more than one ultrasonic transducer 507 as transmitters and transmitting a pulse (or a modulated pulse) from the external transmitters 507, it is possible to detect time differences between the two or more coming signals. Taking into account the velocity of sound in the tissues, it is possible to estimate the relative distance of the capsule 550 from the transmitters 507 and hence calculate if there is a change in position of the capsule 550. This information can be to sense if there is movement and hence, an algorithm in the capsule 550 can use this information to trigger capsule scanning due to movement.

In another embodiment of this invention, the capsule 550 also calculates the change in position of the capsule 550. These changes related to position movement are used by the capsule controller 502 to decide when to start and stop scanning, that is, illumination by the capsule 550. Additionally, relative position information which is correlated to ultrasonic time difference detected on board the capsule 550 can be transmitted from the capsule 550 to an external receiver (not shown) or stored in the capsule 550 for later analysis of the position of the capsule in time relative to the external transmitter 507 on the patient 501.

It should be appreciated that the calculation as to whether the capsule 550 has moved is based on a correlation of at least one parameter coming from at least one sensor such as a pressure sensor, an accelerometer, an alternating magnetic detector, an ultrasonic detector and other parameters coming from the imaging data that the capsule 550 generates.

Referring again to FIG. 5A, in another exemplary embodiment of a screening system a method of detecting motion of a capsule within the GI tract of a patient and activating the capsule only when such movements occur may include using a radio frequency (RF) system, rather than an ultrasound system, to sense change in orientation and/or position of the capsule in the colon and hence capsule movement. For example, the capsule 550 may include at least one RF antenna and an electronic circuit that is communicable with the antenna to pick up RF signals generated by an external RF transmitter, or a number of RF transmitter antennae, carried on a belt or a vest worn by the patient, or are otherwise attached to the patient by an adhesive sticker. The antenna(e) is positioned so that it is securely attached to the patient and moves with the patient, thus defining the antenna in the patient system of coordinates.

Consequently, when the capsule in the colon starts to move, a change in relative orientation of the capsule will change the detected amplitude of the RF transmission received by the RF transducers within the capsule 550. This change in relative amplitude received by the capsule transducers can signify change in orientation of the capsule 550, which can be linked to capsule movement in the colon 505. In turn, the processor 502 will identify relative changes in the detected RF emissions over time and generate a second output associated with detected movement of the capsule within the GI tract. The processor/controller 502 in the capsule 550 can use this information to decide when to start and stop scanning by the capsule, thereby limiting scanning radiation emissions to the adjacent body tissue only when needed, such as when the capsule is moving within the GI tract.

Additionally, the calculation as to whether the capsule has moved can also be based upon a correlation of more than one parameter sensed by multiple sensors incorporated into the capsule, such as a pressure sensor, an accelerometer, RF power, an alternating magnetic detectors, and other parameters coming from the imaging data that the capsule generates.

According to some aspects, an RF signal is transmitted from the capsule in sync with an alternating electromagnetic signal transmitted from at least one coil in the capsule. On the patient, an RF receiver antenna or a number of RF receiver antennae are placed on a belt, vest or attached to the patient by an adhesive sticker. The antenna(e) is connected to an RF receiver. RF signals received on the patient are used to sync the electronic circuits that are connected to at least one coil or other magnetic sensor positioned so that they are securely attached to the patient and move with the patient, thus defines them in the patient system of coordinates. In this way, feeble electromagnetic signals picked up by the magnetic sensor can be detected with improved sensitivity since the exact timing of each such signal is known synced with the RF signal. This allows improved detection since noise picked up at times in which there is no RF transmission, hence no electromagnetic transmission can be ignored.

According to some aspects, the exemplary capsule serves as the receiver, and the transmissions of the electromagnetic signals are transmitted from the coils placed on the patient belt, sticker, etc. in some aspects, an RF signal is transmitted from the external receiver in sync with an alternating electromagnetic signal transmitted from at least one coil on the patient belt or vest. On the patient, an RF transmitting/receiving antenna or a number of RF transmitting/receiving antennae are placed on a belt, vest or attached to the patient by an adhesive sticker. The antenna(e) is connected to an RF transceiver. RF signals received in the capsule are used to sync the electronic circuits that are connected to at least one coil or other magnetic sensor in the capsule. In this way, feeble electromagnetic signals picked up by the magnetic sensor in the capsule can be detected with improved sensitivity since the exact timing of each such signal is known synced with the RF signal. This allows improved detection since noise picked up at times in which there is no RF transmission, hence no electromagnetic transmission can be ignored.

In accordance with another embodiment of the disclosure, a process is used for compensation of movements between the coils and other magnetic sensors on the patient's belt or vest or stickers to minimize artifacts due to such movements. Referring to FIGS. 2A-4B, a capsule 250, 350, 450 is sized and adapted to be swallowed by a patient for diagnostic or treatment purposes. The capsule may incorporate at least one coil and an electronic circuit that is in communication with the coil to pick up signals from the coil. On the patient, at least two coils are placed on a belt or vest or are attached to the patient by an adhesive sticker. The coils are positioned so that they are well attached to the patient and move with the patient, thus defining the coils the patient system of coordinates.

During the process of signal acquisition, the coils on the patient transmit, each on its turn, an electromagnetic signal while the other coils are configured to pick up this signal. In the controller of the external unit on the patient, a table of signal values may be stored whereby for each coil emitting the electromagnetic signal, the values of the signals picked up by all other coils are stored. This process may be done in a cyclic fashion at short intervals for all coils. Transmissions from the capsule are done at time intervals when the coils on the patient body are not transmitting. These values are stored in a memory and used as a reference to ascertain whether movements between the coils have occurred. This process ensures that movements due to change in position between coils on the patient body are not mistaken as capsule movements since such movements will be detected separately from change in magnetic signals due to capsule movements.

In another embodiment of the disclosure, a low pass filter or a Kalman filter is used to continuously update the change in signals in the table due to position change between the coils on the patient body. These values are stored in a memory and used as a reference to ascertain whether movements between the coils have occurred. This process ensures that movements due to change in position between coils on the patient body are not mistaken as capsule movements since such movements will be detected separately from change in magnetic signals due to capsule movements.

According to various aspects, accelerometers and/or gyros are placed near the coil (or other electromagnetic sensor) on the patient body on a belt or a vest or by way of stickers. A control unit is connected to these accelerometers and/or gyros and is configured to calculate the relative movements of each coil (or other electromagnetic sensor) on the patient body. In case one or more than one of the coils (or other electromagnetic sensor) are moving relative to each other as detected by the accelerometers and or gyros attached to them, the controller will take that into account when sensing possible changes in the electromagnetic signal pickup from the sensors so that relative movements of the electromagnetic sensors between themselves are not mistaken for capsule movements.

In another embodiment of this invention, ultrasonic transducers (such as piezoelectric transducers, magneto constrictive transducers or others) are placed near the coil (or other electromagnetic sensor) on stickers placed on the patient. A control unit is connected to these ultrasonic transducers and is configured to measure distance between the transducers based on time of flight of the acoustic signals, whereby these transducers serve both as transmitters and receivers of the acoustic signals. The controller then calculates the relative distance between the transducers and hence the relative movements of each coil (or other electromagnetic sensor) on the patient body. In case one or more than one of the coils (or other electromagnetic sensor) are moving relative to each other as detected by ultrasonic transducers attached to them, the controller will take that into account when sensing possible changes in the electromagnetic signal pickup from the sensors so that relative movements of the electromagnetic sensors between themselves are not mistaken for capsule movements.

It will be apparent to those skilled in the art that various modifications and variations can be made in the medical devices and methods of the present invention without departing from the scope of the invention. Other embodiments of the invention will be apparent to those skilled in the art from consideration of the specification and practice of the invention disclosed herein. It is intended that the specification and examples be considered as exemplary only.

What is claimed is:

1. A system for detecting clinically-relevant features of a gastrointestinal (GI) tract of a subject, comprising:
an external magnetic field generator adapted to be affixed to a patient for generating at predetermined time intervals a magnetic field with respect to the subject, said magnetic field having a predetermined polarity and flux density, thereby establishing a patient coordinate system based upon the magnetic field; and
a capsule with a capsule housing, said capsule adapted to be swallowed by a subject, said capsule including:
at least one radiation source emitting X-ray or gamma radiation; at least one radiation detector configured to detect in a first energy window collimated X-ray fluorescence radiation from the X-ray contrast agent composition excited by the emitted radiation, and to detect a second energy window Compton-backscattered radiation from the X-ray contrast agent and the wall of the GI tract produced in response to the emitted radiation;
a magnetic field detector configured to generate a first output responsive to a detected polarity and detected levels of flux density during exposure to the externally generated magnetic field;
a processor configured to generate a second output based upon relative changes in the first output, said first output varying as a function of relative changes in physical location or angular orientation of the capsule associated with movement of the capsule within the GI tract; and
a control unit configured to selectively enable or disable X-ray or gamma ray emissions from the capsule produced by the at least one radiation source based upon the second output, such that the GI tract is selectively irradiated upon detected movement of the capsule.

2. The system according to claim 1, wherein the at least one radiation source comprises an X-ray generator and a power source, and the control unit selectively enables or disables radiation emissions by regulating power supplied to the X-ray generator based upon the second output.

3. The system according to claim 1, wherein the at least one radiation source comprises a radioisotope selected from the group comprising T1201, Xe133, Hg197, Yb169, Ga67, Tc99, Tc99m, In111, I131 and Pd100.

4. The system according to claim 3, wherein the control unit further comprises a power source, at least one radiation shield movably configured with respect to the at least one radiation source, and a motor configured to controllably reposition the at least one radiation shield relative to the at least one radiation source, such that the GI tract is selectively irradiated based upon the second output.

5. The system according to claim 1, wherein the magnetic field detector comprises a magnetic compass.

6. The system according to claim 2, wherein the external magnetic field generator comprises at least two magnets affixed at spaced apart locations on a belt to be worn by the subject.

7. The system according to claim 3, wherein the at least two magnets comprise electromagnets and a controlled current generator.

8. The system according to claim 4, wherein the controlled current generator varies the current supplied to each of the at least two electromagnets.

9. A system for detecting clinically-relevant features of a gastrointestinal (GI) tract of a subject, comprising:
an external signal transmitter adapted to be affixed to a patient for generating at predetermined time intervals a transmission field with respect to the subject, said transmission field having a predetermined amplitude and frequency, thereby establishing a patient coordinate system based upon the transmission field; and
a capsule with a capsule housing, said capsule adapted to be swallowed by a subject, said capsule including:
at least one radiation source emitting X-ray or gamma radiation; at least one radiation detector configured to detect in a first energy window collimated X-ray fluorescence radiation from the X-ray contrast agent composition excited by the emitted radiation, and to detect a second energy window Compton-backscattered radiation from the X-ray contrast agent and the wall of the GI tract produced in response to the emitted radiation;
a transmission field detector configured to generate a first output responsive to a detected amplitude and frequency during exposure to the externally generated transmission field;
a processor configured to generate a second output based upon relative changes in the first output, said first output varying as a function of relative changes in physical location or angular orientation of the capsule associated with movement of the capsule within the GI tract; and
a control unit configured to selectively enable or disable X-ray or gamma ray emissions from the capsule produced by the at least one radiation source based upon the second output, such that the GI tract is selectively irradiated upon detected movement of the capsule.

10. The system according to claim 9, wherein the external signal transmitter comprises at least one transducer configured to generate radio frequency emissions.

11. The system according to claim 10, wherein the transmission field detector further comprises at least one antenna configured to receive said radio frequency emissions.

12. The system according to claim 9, wherein the external signal transmitter comprises at least one transducer configured to generate ultrasound emissions.

13. The system according to claim 12, wherein the transmission field detector further comprises at least one transducer configured to receive said ultrasound emissions.
